SEQUENCE LISTING (14 sequences)

SEQ ID NO: 1 - protein, 233 aa
FLIGNLALSDVLMCTACVPLTLAYAFEPRGWVFGGGLCPLVFFLQPVTVYVSVFTLTTIAVDRYVVLVHPLRRRISLRLSAYAVLAIWALSAVLALPAAVHTYHAELKPHDVRLCEEFWGSQERQRQLYAWGLPLVTYLLPLLVILLSYVRVSVKLRNRVVPDCVTQSQADWDRARRRRTFCLLVVVVVVFAVCWLPLHVFNLLRDLDPHAIDPYAFGLVQLLCHWLAMSSAC

SEQ ID NO: 2 - DNA (other nucleic acid), 34 bp; single-stranded, linear
  misc_feature one-of(14, 16, 25): /standard_name= “N = Inosine”
  description: /desc = “Synthetic”
acaatattamaarnrnatgmgramngtnacsaac

SEQ ID NO: 3 - DNA (genomic), 701 bp; single-stranded, linear
ttcctcatcggcaacctggccttgtccgacgtgctcatgtgcaccgcctgcgtgccgctcacgctggcctatgccttcgagccacgcggctgggtgttcggcggcggcctgtgccccctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtactggcgctgcccgccgccgtgcacacctatcacgcggagctcaagccgcacgacgtgcgcctctgcgaggagttctggggctcccaggagcgccagcgccagctctacgcctgggggctgccgctggtcacctacctgctccctctgctggtcatcctcctgtcttacgtccgggtgtccgtgaagctccgcaaccgcgtggtgccggactgcgtgacccagagccaggccgactgggaccgcgctcggcgccggcgcaccttctgcttgctggtggtggtcgtggtggttttcgccgtctgctggctgccgctgcacgtcttcaacctgctgcgggacctcgacccccacgccatcgacccttacgcctttgggctggtgcagctgctctgccactggctcgccatgagttcggcctgcta

SEQ ID NO: 4 - protein, 78 aa
ARPTGTALGRRRTFCLLVVVVVVFAVCWLPLHVFNLLRDLDPHAIDPYAFGLVQLLCHWLAMSSACYNPIFYGFLKGL

SEQ ID NO: 5 - DNA (genomic), 237 bp; single-stranded, linear
aagccaggccgactgggaccgcgctcgggcgccggcgcaccttctgcttgctggtggtggtcgtggtggttttcgccgtctgctggctgccgctgcacgtcttcaacctgctgcgggacctcgacccccacgccatcgacccttacgcctttgggctggtgcagctgctctgccactggctcgccatgagttcggcctgctacaatcccatcttctatggcttcctcaaaggcctgt

SEQ ID NO: 6 - DNA (genomic), 1173 bp; single-stranded, linear
gaattcggcttcggactttgattacctttgaacaggtggccatggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaagggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccttgagttcggcctgctacaaccccttcatctacgcctggctgcacgacagcttccgcgaggagctgcgcaaactgttggtcgcttggccccgcaagatagccccccatggccagaatatgaccgtcagcgtggtcatctgatgccacttaaagccgaattc

SEQ ID NO: 7 - DNA (genomic), 1174 bp; single-stranded, linear
gaattcggcttcggactttgattacctttgaacaggtggtcatggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtagtgatcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatctgatgccacttaaagccgaattc

SEQ ID NO: 8 - DNA (genomic), 1174 bp; single-stranded, linear
gaattcggcttcggactttgattacctttgaacaggtggccatggcctcatcgaccactcggggccccagggtttctgacttattttctgggctgctgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagtgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggccacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatctgatgccacttaaagccgaattc

SEQ ID NO: 9 - DNA (genomic), 1174 bp; single-stranded, linear
gaattcggcttcggactttgattacctttgaacaggtggccatggcctcatcgaccactcggggccccagggtttctgacttattttctgggctgccgccggcggacacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccactgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatctgatgccacttaaagccgaattc

SEQ ID NO: 10 - DNA (genomic), 1152 bp; single-stranded, linear
cggactttgattacctttgaacaggtggccatggcctcatcgaccactcggggccccagggtttctgacttattttctgggctgccgccggcggtcacaactcccgccaaccagagcgcagaggcctcggcgggcaacgggtcggtggctggcgcggacgctccagccgtcacgcccttccagagcctgcagctggtgcatcagctgaaggggctgatcgtgctgctctacagcgtcgtggtggtcgtggggctggtgggcaactgcctgctggtgctggtgatcgcgcgggtgcgccggctgcacaacgtgacgaacttcctcatcggcaacctggccttgtccgacgtgctcatgtgcaccgcctgcgtgccgctcacgctggcctatgccttcgagccacgcggctgggtgttcggcggcggcctgtgccacctggtcttcttcctgcagccggtcaccgtctatgtgtcggtgttcacgctcaccaccatcgcagtggaccgctacgtcgtgctggtgcacccgctgaggcggcgcatctcgctgcgcctcagcgcctacgctgtgctggccatctgggcgctgtccgcggtgctggcgctgcccgccgccgtgcacacctatcacgtggagctcaagccgcacgacgtgcgcctctgcgaggagttctggggctcccaggagcgccagcgccagctctacgcctgggggctgctgctggtcacctacctgctccctctgctggtcatcctcctgtcttacgtccgggtgtcagtgaagctccgcaaccgcgtggtgccgggctgcgtgacccagagccaggccgactgggaccgcgctcggcgccggcgcaccttctgcttgctggtggtggtcgtggtggtgttcgccgtctgctggctgccgctgcacgtcttcaacctgctgcgggacctcgacccccacgccatcgacccttacgcctttgggctggtgcagctgctctgccactggctcgccatgagttcggcctgctacaaccccttcatctacgcctggctgcacgacagcttccgcgaggagctgcgcaaactgttggtcgcttggccccgcaagatagccccccatggccagaatatgaccgtcagcgtggtcatctgatgccactta

SEQ ID NO: 11 - protein, 380 aa
RTLITFEQVAMASSTTRGPRVSDLFSGLPPAVTTPANQSAEASAGNGSVAGADAPAVTPFQSLQLVHQLKGLIVLLYSVVVVVGLVGNCLLVLVIARVRRLHNVTNFLIGNLALSDVLMCTACVPLTLAYAFEPRGWVFGGGLCHLVFFLQPVTVYVSVFTLTTIAVDRYVVLVHPLRRRISLRLSAYAVLAIWALSAVLALPAAVHTYHVELKPHDVRLCEEFWGSQERQRQLYAWGLLLVTYLLPLLVILLSYVRVSVKLRNRVVPGCVTQSQADWDRARRRRTFCLLVVVVVVFAVCWLPLHVFNLLRDLDPHAIDPYAFGLVQLLCHWLAMSSACYNPFIYAWLHDSFREELRKLLVAWPRKIAPHGQNMTVSVVI

SEQ ID NO: 12 - DNA (other nucleic acid), 35 bp; single-stranded, linear
  misc_feature one-of(13, 17, 23, 26): /standard_name= “N = Inosine”
  description: /desc = “Synthetic”
acaggcctttsanrmancmrtanawnatgggrttg

SEQ ID NO: 13 - DNA (other nucleic acid), 22 bp; single-stranded, linear
  description: /desc = “Synthetic”
cggactttgattacctttgaac

SEQ ID NO: 14 - DNA (other nucleic acid), 21 bp; single-stranded, linear
  description: /desc = “Synthetic”
taagtggcatcagatgaccac

(19) United States
(12) Patent Application Publication
(10) Pub. No.: US 2004/0110185 A1
(43) Pub. Date: Jun. 10, 2004

(54) Human hypothalmic ("HR") receptor polypeptide compositions, methods and uses thereof

(75) Inventor: David Duhl, Oakland, CA (US)

Correspondence Address: Chiron Corporation, Intellectual Property - R440, P.O. Box 8097, Emeryville, CA, 94662-8097, US

(73) Assignee: Chiron Corporation, Emeryville, CA (US)

(21) Appl. No.: 10/611,210

(22) Filed: Jun. 30, 2003

Related U.S. Application Data
(63) Continuation of application No. 09/000,193, filed on Jan. 27, 1998, now abandoned.
Application No. 09/000,193 is a 371 of international application No. PCT/US96/13974, filed on Aug. 29, 1996.
(60) Provisional application No. 60/003,003, filed on Aug. 31, 1995.
(60) Provisional application No. 60/003,039, filed on Aug. 29, 1995.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

A new human hypothalmic receptor has been identified, and the amino acid and nucleotide sequence of the receptor are provided. The nucleotide sequence is useful to construct expression cassettes and vectors to produce host cells which are capable of expressing the receptor, its mutants, fragments, or fusions. Such polypeptides are useful for identifying new agonists and antagonists.

### DESCRIPTION

1. Technical Field

This invention relates to the fields of molecular biology and pharmaceutical research. More specifically, this invention relates to the identification of a new human receptor polypeptide and nucleic acids encoding the polypeptide as well as vectors and host cells for producing such. This invention also relates to the use of the new receptor polypeptides to measure ligand binding, signal transduction and identification of new receptor agonists and antagonists. The new amino acid and nucleic acid sequences described herein permit production of mutant, fragment and fusion polypeptides of the native human receptor polypeptide. The invention also relates to antibodies to these polypeptides and the methods of production of the polypeptides, nucleic acids, vectors and host cells.

2. Background of the Invention

Neuropeptide receptors are implicated in neurotransmitter interactions and can modulate neurotransmitter levels. This class of receptors include neuropeptide Y (“NPY”), somatostatin (“SS”), tachykinin (“TX”), and cholecystokinin (“CCK”) receptors.

These receptors are members of the seven-transmembrane receptor family. This type of receptor contains seven helical domains which span the cell membrane. These seven transmembrane regions are linked by three intracellular and three extracellular loops; in addition, these receptors each possesses an extracellular amino terminal tail and an intracellular carboxyl terminal tail.

The extra- and intracellular loops contribute to the ligand binding and the signal transduction activity of the receptor. For example, the intracellular loops of the receptor are known to be bind to guanyl-nucleotide-binding proteins, or G-proteins. G-proteins interconvert between GDP- and GTP-binding forms. Seven transmembrane receptors are also known as G-protein coupled receptors.

Binding of ligand to the receptor triggers the conversion of the G-protein to its GTP-binding form, which initiates the cascade of reactions to generate the desired biological response. This cascade is called signal transduction. Signal transduction activity can be detected measuring various reactions. For example, signal transduction of some seven-transmembrane receptors causes an increase of intracellular Ca²⁺ levels and activation of phospholipase C. Signal transduction of other seven-transmembrane receptors can be measured by observing the levels of inositol triphosphate (IP₃) and diacylglycerol (DAG). Signal transduction of other receptors can modulate the levels of adenosine cyclic 3′,5′-monophosphate (cAMP). Though the role of the G-proteins has been elucidated, the intracellular loop interactions with these proteins and with other proteins are unknown.

Welch et al., Biochem. Biophys. Res. Comm. 209(2: 606-613 (1995), isolated cDNA from rat hypothalmus that was reported to encode a fragment of seven transmembrane receptor. The authors noted that the encoded polypeptide fragment may be related to the neuropeptide receptor family. The authors also published a paper reporting a human receptor sequence. See Marchese et al., Genomics 29: 335-344 (1995).

### DISCLOSURE OF THE INVENTION

The inventors herein have identified a new human seven-transmembrane receptor that comprises an unique amino acid sequence. The native human receptor is referred herein as the “human hypothalmic receptor” or “hHR.”

It is an object of the invention to provide a polynucleotide encoding a human hypothalmic receptor polypeptide. The polypeptide of the invention comprises an amino acid sequence which exhibits substantially sequence identity to SEQ ID NO:11 or fragment thereof SEQ ID NO:11 is the consensus sequence constructed according to Example 2. The polynucleotide of the invention will be substantially free of polynucleotide that do not encode human HR polypeptides.

Polynucleotides of the present invention also include those obtainable as follows:

(a) isolating mRNA from human cells that contains hHR polypeptide;

(b) producing cDNA template therefrom;

(c) amplifying a portion of the the cDNA template using

a first polynucleotide primer, the sequence of the primers encodes at least three consecutive amino acids of SEQ ID NO:11 and using

a second polynucleotide primer, the reverse complement of the sequence of the second primer encodes at least three consecutive amino acids of SEQ ID NO:11,

wherein the first primer sequence is different from the second primer sequence; and

(d) obtaining the amplified polynucleotide fragment.

Polynucleotides of the present invention also include a polynucleotide hybridizable under stringent conditions to a sequence encoding a polypeptide comprising an amino acid sequence exhibiting substantially sequence identity to SEQ ID NO:11 or fragment thereof containing at least eight consecutive amino acids residues.

It is another object of the invention to provide an expression cassette comprising a promoter operably linked to a human HR polypeptide coding sequence.

Yet another object of the invention is to provide a cell capable of producing a human HR polypeptide, wherein the cell comprises an expression cassette.

Another object of the invention is a method of producing human HR polypeptide comprising culturing a cell having an expression cassette under conditions inducing expression.

It is another object of the invention is a polypeptide produced by a cell having an expression cassette under conditions inducing expression.

Yet another object of the invention is a polypeptide encoded by the polynucleotides of the invention. These human HR polypeptides include mutants, fragments, and fusions as well as the native human HR. The polypeptides are substantially free of other human cell components, such as intracellular proteins.

Another object of the invention are antibodies that bind specifically to human HR polypeptides.

It is another object of the invention to provide a method to screen for candidates that are capable of binding to human HR polypeptides. The method comprises:

(a) providing a human HR polypeptide substantially free of other human intracellular components;

(b) exposing the human HR polypeptide to the candidate under conditions that permit the polypeptide and the candidate to bind and form a complex;

(c) measuring the amount of complex was formed.

Yet another object of the invention is to provide a method to screen for candidate that are capable of triggering human HR signal transduction activity. The method comprises:

(a) providing a cell producing a human HR polypeptide;

(b) exposing said produced hHR polypeptide to a substrate;

(c) measuring hHR polypeptide signal transduction activity.

Another object of the invention is to provide a method of measuring human HR signal transduction activity.

(a) providing a cell producing a human HR polypeptide;

(b) exposing said produced hHR polypeptide to a substrate;

(c) measuring hHR polypeptide signal transduction activity.

Yet another object of the invention is to provide a method to detect polynucleotides encoding human HR polypeptides. The method comprises:

(a) providing a nucleic acid probe which hybridizes to SEQ ID NO:10;

(b) hybridizing a sample of polynucleotides to said probe to form a duplex; and

(c) detecting said duplexes.

SEQ ID NO:10 is the polynucleotide consensus sequence constructed according to Example 2.

These objects, features, and advantages are achieved by the present invention.

Modes of Carrying Out the Invention

A. Definitions

As used herein, the term “human hypothalmic receptor” of“hHR” refers to the native polypeptides found in nature and includes allelic variants that possess substantially the same biological activity. One example is a polypeptide comprising an amino acid sequence of SEQ ID NO:11. The amino acid sequence of the native receptor will comprise a sequence that varies slightly; typically, by less than about 10-20 amino acids from the presently described hHR, a partial sequence of which is show in SEQ ID NO:11.

“Human hypothalmic receptor polypeptides” include mutants, fragments, and fusions of the native human HR as well as the native human HR. These polypeptides comprise an amino acid sequence that exhibits substantial sequence identity to SEQ ID NO:11 or a fragment thereof. These polypeptides will retain more than about 80% amino acid identity with SEQ ID NO:11 of fragment thereof; more typically, more than about 85%; even more typically, at least 90%. Preferably, these polypeptides will exhibit more than about 92% amino acid sequence identity with SEQ ID NO:11 or fragment thereof; more preferably, more than about 94%; even more preferably, more than about 96%; even more preferably, more than about 98%; even more preferably, more than about 99%. All of these polypeptides will exhibit either immunological, ligand binding, or signal transduction properties of the native human HR. For example, human HR polypeptides can exhibit at least about 20% ligand binding or signal transduction activity of the native human hypothalmic receptor. More typically, the polypeptides exhibit at least about 40%, even more typically the polypeptides exhibit at least about 60% of the native human HR ligand binding or signal transduction activity. The human HR polypeptides herein can exhibit immunological properties of the native human HR, in which case an antibody to the native hHR bind specifically with the human HR polypeptides.

“Signal transduction activity” occurs when ligand binding to the human HR polypeptide triggers a specified biological response in a cell or cell extract. The biological response is the result of a cascade of biochemical reactions. Measurement of any one of these reactions can indicate that the desired biological response was triggered. For example, hypothalmic receptor is a G-coupled protein which, when proper signal transduction activity occurs, can modulate intracellular levels of Ca²⁺, IP₃, DAG, or cAMP. An assay for the measurement of increased levels of free cytosolic Ca²⁺ is described in Sakurai et al., EP 480 381, and Adachi et al., FEBS Lett 311(2): 179-183 (1992). Intracellular IP₃ concentrations can be measured according to Sakurai et al., EP 480 381 and Amersham's inositol 1,4,5-trisphosphate assay system (Arlington Heights, Ill., U.S.A.). Levels of cAMP can be measured according to Gilman et al., Proc Natl Acad Sci 67: 305-312 (1970). In addition, a kit for assaying levels of cAMP is available from Diagnostic Products Corp. (Los Angeles, Calif., U.S.A.). These assays can be effective for determining hypothalmic receptor signal transduction activity whether the receptor is normally expressed by the cell or expressed by a heterologous cell type by recombinant techniques.

Proper signal transduction activity depends not only on receptor/ligand binding but also depend on the presence of certain intracellular proteins. Thus, though a number of cells are capable, via recombinant techniques, of expressing hypothalmic receptor polypeptides, no biological response will be detected despite proper receptor/ligand binding if the host cell does not produce the needed intracellular proteins. Signal transduction activity can be detected in cells that are known to express the hypothalmic receptor in humans, such as heart, lung, brain, and placental cells. Heterologous host cells, COS and Chinese Hamster Ovary (CHO) cells, for instance, can trigger the desired biological response if altered to produce the receptor by recombinant techniques.

A composition containing A is “substantially free of” B when at least 85% by weight of the total A+B in the composition is A. Preferably, A comprises at least about 90% by weight of the total of A+B in the composition, more preferably at least about 95% or even 99% by weight.

A promoter herein is “heterologous” to a coding sequence if the promoter is not operably linked to the coding sequence in nature. A “native” promoter is operably linked to the coding sequence in nature.

An “origin of replication” is a DNA sequence that initiates and regulates replication of polynucleotides, such as an expression vector. The origin of replication behaves as an autonomous unit of polynucleotide replication within a cell, capable of replication under its own control. With certain origins of replication, an expression vector can be reproduced at a high copy number in the presence of the appropriate proteins within the cell. Examples of origins are the 2μ and autonomously replicating sequences, which are effective in yeast; and the viral T-antigen, effective in COS-7 cells.

Host cells capable of producing hypothalmic receptor polypeptides are cultured “under conditions inducing expression.” Such conditions allow transcription and translation of the DNA molecule encoding the hypothalmic receptor polypeptide. These conditions include cultivation temperature, oxygen concentration, media composition, pH, etc. For example, if the trp promoter is utilized in the expression vector, the media will lack tryptophan to trigger the promoter and induce expression. The exact conditions will vary from host cell to host cell and from expression vector to expression vector.

A nucleic acid molecule is said to “hybridize” with a target polynucleotide sequence if the molecule can form a duplex or double stranded complex with that target, which is stable enough to be detected. Hybridization of a nucleic acid molecule to a target polynucleotide depends on (1) the sequence of the nucleic acid molecule and (2) the hybridization conditions. The sequence of the molecule need not be exactly complementary to the target polynucleotide. For example, a non-complementary nucleotide sequence may be attached to the 5′ end of the molecule, with the remainder of the sequence being complementary to target polynucleotide. Alternatively, non-complementary bases or longer sequences can be interspersed into the nucleic acid molecule, provided that the sequence has sufficient complementarity with target polynucleotide to hybridize with the target and thereby form a duplex that can be detected. The exact length and sequence of the molecule will depend on the hybridization conditions, such as temperature, salt condition and the like. For example, for diagnostic applications, depending on the complexity of the analyte sequence, the nucleic acid probe typically contains 15-25 or more nucleotides, although it may contain fewer nucleotides. Short primers generally require cooler temperatures to form sufficiently stable hybrid complexes with the template. Strigent hybridization conditions will vary depending of the length and complementarity of the probe and target sequence.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, altered antibodies, univalent antibodies, the Fab proteins, and single domain antibodies. Antibodies do not possess the signal transduction activity of hypothalmic receptor polypeptides.

An antibody “differentiates” human HR polypeptides from native rat HR when the antibody has a higher binding affinity for the human HR polypeptides than for the native rat HR. Binding affinity can be measured typically using ELISA or RIA formats.

B. General Method

This invention provides the amino acid and nucleotide sequence of a novel human hypothalmic receptor. With these disclosed sequences, nucleic acid probe assays and expression cassettes and vectors for hypothalmic receptor polypeptides can be produced. The expression vectors can be transformed into host cells to produce hypothalmic receptor polypeptides. The purified polypeptides can be used to produce antibodies to distinguishes rat hypothalmic receptors from human hypothalmic receptor polypeptides. Also, the host cells or extracts can be utilized for biological assays to isolate agonists or antagonists.

Nucleic Acid Hypothalmic Receptor Probe Assays

Expression of human hypothalmic receptor mRNA is found in, but not limited to, brain and placental cells. Variation of mRNA levels in different cell types can be exploited with nucleic acid probe assays. For example, PCR, branched DNA probe assays, or blotting techniques can utilize nucleic acid probes substantially identical or complementary to a sequence encoding at least 3 or 4 consecutive amino acid residues of SEQ ID NO:11. With these probes and the assays can determine the presence or absence of hypothalmic cDNA or mRNA.

Using nucleic acid probe assays, polynucleotide probes will hybridize a sequence encoding a polypeptide comprising an amino acid sequence exhibiting substantially sequence identity to SEQ ID NO:11 or fragment thereof Though many different nucleotide sequences will encode human HR polypeptides, SEQ ID NO:10 is preferred to detect cDNA or mRNA isolated from human cells because it is the actual sequence isolated from human cells having human HR polypeptides. Because cDNA is complementary to mRNA, for cDNA detection, the nucleic acid probe will hybridize complement of SEQ ID NO:10. In contrast, for mRNA detection, the nucleic acid probe will hybridize to SEQ ID NO:10, itself. The nucleic acid probe sequences need not be identical to SEQ ID NO:10 or its complement. Some variation in the sequence and length can lead to increased assay sensitivity if the nucleic acid probe can form a duplex with target nucleotides, which can be detected. Additional non-hypothalmic receptor sequence may be helpful as a label to detect the formed duplex.

Probes of at least 15 nucleotides; more preferably, at least 20 nucleotides; even more preferably, at least 30 nucleotides, are useful in the nucleic acid probe assays described below.

These probes may be produced by synthetic procedures, such as the triester method of Matteucci et al. (J. Am. Chem. Soc. (1981) 103:3185), or according to Urdea et al. Proc. Natl. Acad. Sci. USA 80: 7461 (1983), or using commercially available automated oligonucleotide synthesizers.

One example of a nucleotide hybridization assay is described in Urdea et al., PCT WO92/02526 and Urdea et al., U.S. Pat. No. 5,124,246, herein incorporated by reference. The references describe an example of a sandwich nucleotide hybridization assay. The described assay utilizes a microtiter plate as a solid support and five sets of oligonucleotides to detect the target sequences. The five oligonucleotide sets are:

(1) plate binding oligonucleotides (oligonucleotide attached to the solid phase in Urdea et al.),

(2) capture oligonucleotides (“capture probes” in Urdea et al.),

(3) labeled probes (“amplifier probes” in Urdea et al.),

(4) branched amplifier oligonucleotides (“multimer” in Urdea et al.), and

(5) enzyme-linked oligonucleotides (“labeled oligonucleotide” in Urdea et al.).

A microtiter plate is coated with the plate binding oligonucleotides (1). These plate binding oligonucleotides contain a sequence that is complementary to a sequence on the capture oligonucleotides (2). The capture oligonucleotides also comprise a second sequence that can hybridize to the target nucleic acids. Via the plate binding and capture oligonucleotides, the target nucleic acids are immobilized to the microtiter plate and separated from unwanted and unbound nucleotides by simply washing the plate.

The target nucleic acids are detected via a labeled probe (3). For this specific assay, the labeled probe comprises a region complementary to the target nucleic acids and region(s) complementary to a region on the branched amplifier oligonucleotides (4). The branched amplifier oligonucleotide comprises multiple regions, which hybridize with a region on the enzyme-linked oligonucleotides (5). The enzyme-linked oligonucleotides cleave light producing molecules that can be detected with a luminometer.

Alternatively, the Polymerase Chain Reaction (PCR) is another well-known means for detecting small amounts of target nucleic acids. The assay is described in Mullis et al., Meth. Enzymol. 155: 335-350 (1987); U.S. Pat. No. 4,683,195; and U.S. Pat. No. 4,683,202, incorporated herein by reference. This method, unfortunately, cannot quantitate the amount of target nucleic acids.

Two “primer” polynucleotides hybridize with the target nucleic acids and are used to prime the reaction. The primers may be composed of sequence, such as restriction sites, that does not encode hHR polypeptides as well as hHR specific sequence. Typically, the primers will include sequence or reverse complement sequence that encodes at least 3 or 4 consecutive amino acid residues of SEQ ID NO:11. However, the primers need not hybridize to a sequence encoding at least 3 or 4 consecutive amino acid residues of SEQ ID NO:11 or its complement. Preferably, the primers are from about 16 to 27 nucleotides in length

PCR reaction comprises of repeating cycle of varying temperatures to (1) melt any double stranded polynucleotide duplexes, (2) permit the primer to anneal to the template; and (3) permit the polymerase to create a new polynucleotide from the primers and template.

Typically, the melting temperature is between about 90° C. and about 100° C.; more typically, between about 92° and about 96° C.; even more typically, about 94° C. The PCR sample is typically incubated at the melting temperature for at least about 15 seconds; even more typically, for at least 30 seconds.

Usually, the annealing temperature is calculated from the nucleotide composition of the primers. One example is four degrees Celsius is tabulated for every G or C nucleotide in the primer, and two degrees is tabulated for every T or A in the primer. The annealing temperature is the sum of the degrees tabulated for all nucleotide in the primer. Typically, the difference of the annealing temperature of any PCR primers are less than about 6 degrees, more typically, less than about 4 degreess, even more typically, equal to or less than about 2 degrees. Preferably, the annealing temperature is between about 50° C. and about 70° C.

Usually, the PCR sample is incubated at the annealing temperature for about 30 seconds, more usually, 1 minute.

Preferably, the extension temperature is between about 65° C. and 72° C., more preferably, betweenn about 68° C. and about 70° C.; even more preferably, 68° C. or 72° C. Typically, the PCR sample is incubated at the extension temperature for about 1 minute, more typically, about 2 minutes.

The cycle of melting, annealing, and extending is repeated between 20 and 50 times; more typically, between 25 and 40 times; even more typically, 30 times.

A thermostable polymerase can creates copies of target nucleic acids from the primers using the original target nucleic acids as a template. After a large amount of target nucleic acids are generated by the polymerase, they can be detected by more traditional methods, such as Southern blots. When using the Southern blot method, the labeled probe will hybridize to a sequence encoding at least 3 or 4 amino acid residues of SEQ ID NO:11 or its complement.

Finally, mRNA or cDNA can be detected by traditional blotting techniques described in Sambrook et al., “Molecular Cloning: A Laboratory Manual” (New York, Cold Spring Harbor Laboratory, 1989). mRNA or cDNA generated from mRNA using a Polymerase enzyme can be purified and separated using gel electrophoresis. The nucleic acids on the gel are then blotted onto a solid support, such as nitrocellulose. The solid support is exposed to a labeled probe for hybridization and then washed to remove any unhybridized probe. Next, the duplexes containing the labeled probe are detected. Typically, the probe is labeled with radioactivity.

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook, et al., MOLECULAR CLONING; A LABORATORY MANUAL, SECOND EDITION (1989), Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 12° to 20° C. below the calculated Tₘ of the hybrid under study. The temperature and salt conditions can often be determined empirically in preliminary experiments in which samples of genomic DNA immobilized on filters are hybridized to the sequence of interest and then washed under conditions of different stringencies. See Sambrook, et al, above at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10-9 to 10-8 μg for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tm=81+16.6(log10Cᵢ)+0.4[%G+C)]−0.6(% formamide)−600/n−1.5(% mismatch).

where Cᵢ is the salt concentration (monovalent ions) and n is the length of the hybrid in base pairs (slightly modified from Meinkoth and Wahl, (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e., stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology and between the probe and the target fragment are not known, the simplest approach is to start with both hybridization and wash conditions which are nonstringent. If nonspecific bands or high background are observed after autoradiography, the filter can be washed at high stringency and reexposed. If the time required for exposure makes this approach impractical, several hybridization and/or washing stringencies should be tested in parallel.

The hybridization techniques and PCR can be used not only for detection of polynucleotides, but also to isolated polynucleotide that code for human HR polypeptides. These polynucleotides can be used to construct vector useful to produce the polypeptides of the invention.

Expression of Human Hypothalmic Receptor Polypeptides

Polynucleotides coding human HR polypeptides can be constructed and can be used to produce human HR polypeptides. The polypeptides can be incorporated in membranes to be used in signal transduction and ligand binding assays. Alternatively, the polypeptides can be used to produce antibodies.

Like genomic DNA, the coding sequence can contain both exons and introns. Exons are the sequences which are translated and encode the desired amino acid sequence. Introns are intervening sequences which are not translated. The coding sequence can contain no introns or multiple exons and introns. The intron sequences are chosen based on convenience. The intron sequence from the native human HR gene can be used or other intron sequences which are recognized by the host cell and will not be translated. Introns are not necessary. The coding sequence, like cDNA, can be free on introns.

Coding sequences can be constructed by synthesizing the desired sequence or by altering a native human HR coding sequence. synthetic genes can be made using codons preferred by the host cell to encode the desired polypeptide. (See Urdea et al., Proc. Natl. Acad. Sci. USA 80: 7461 (1983).) Alternatively, the desired native human HR coding sequence can be cloned from nucleic acid libraries using probes based on the sequence shown in SEQ ID NO:10, for example. Probes or primers used to isolated native human HR polypeptide encoding polynucleotides can include sequence that encode the transmembrane region, cytoplasmic faces that interact with G-proteins, or extracellular ligand binding regions or native human HR polypeptides.

Techniques for producing and probing nucleic acid sequence libraries are described, for example, in Sambrook et al., “Molecular Cloning: A Laboratory Manual” (New York, Cold Spring Harbor Laboratory, 1989). Useful libraries to isolate native human HR polypeptide encoding polynucleotides include brain, hypothalmus, and genomic libraries.

Other recombinant techniques, such as site specific mutagenesis, PCR, enzymatic digestion and ligation, can also be used to construct the desired human hypothalmic receptor polypeptide coding sequence.

The amino acid sequence of human HR polypeptides can be divided into four general categories: mutants, fragments, fusions, and the native human hypothalmic receptor polypeptides. The native human hypothalmic receptor polypeptides are those that occur in nature. The amino acid sequence of native polypeptides will comprise a sequence that varies slightly; typically, less than by 10-20 amino acids from SEQ ID NO:11

A sequence encoding a native human HR can be easily modified to encode other classes of human HR polypeptides. For example, mutants can be constructed by making conservative amino acid substitutions. The following are examples of conservative substitutions: Gly⇄Ala; Val⇄Ile⇄Leu; Asp⇄Glu; Lys ⇄Arg; Asn⇄Gln; and Phe⇄Trp⇄Tyr. A subset of mutants, called muteins, is a group of polypeptides with the non-disulfide bond participating cysteines substituted with a neutral amino acid, generally, with serines. These mutants may be stable over a broader temperature range than native hypothalmic receptor polypeptides. Mutants can also contain amino acid deletions or insertions compared to the native human hypothalmic receptor polypeptides. The coding sequence of mutants can be constructed by in vitro mutagenesis of the native human hypothalmic receptor polypeptide coding sequences.

Fragments are amino and/or carboxyl terminal amino acid deletions of mutant or native human HR polypeptides. The number of amino acids that are truncated is not critical as long as the polypeptide fragment exhibits the desired immunological, ligand binding, or signal transduction property. Fragments need not comprise all seven transmembrane domains, three extracellular loops, three intracellular loops, amino and carboxyl terminal tails. A fragment may only include the amino acid sequence similar to the amino terminal tail, for example. Fragments of interest contain sequence similar to one or more the loops of native human HR. Polypeptide fragments of immunological significance comprise, for example, an epitope not shared by the native rat HR. Such polypeptides may be only 5-15 amino acids in length. Examples of amino acid sequence of fragments include amino acid number 1-8 (aa1 to aa8) of SEQ ID NO:11; aa2 to aa9 of SEQ ID NO:11; aa3 to aa10 of SEQ ID NO:11; aa4 to aa11 of SEQ ID NO:11; aa5 to aa12 of SEQ ID NO:11; aa6 to aa13 of SEQ ID NO:11; aa7 to aa14 of SEQ ID NO:11; aa8 to aa15 of SEQ ID NO:11; aa9 to aa16 of SEQ ID NO:11; aa10 to aa17 of SEQ ID NO:11; aa11 to aa18 of SEQ ID NO:11; aa12 to aa19 of SEQ ID NO:11; aa13 to aa20 of SEQ ID NO:11; aa14 to aa21 of SEQ ID NO:11; aa15 to aa22 of SEQ ID NO:11; aa16 to aa23 of SEQ ID NO:11; aa17 to aa24 of SEQ ID NO:11; aa18 to aa25 of SEQ ID NO:11; aa19 to aa26 of SEQ ID NO:11; aa20 to aa27 of SEQ ID NO:11; aa21 to aa28 of SEQ ID NO:11; aa22 to aa29 of SEQ ID NO:11; aa23 to aa30 of SEQ ID NO:11; aa24 to aa31 of SEQ ID NO:11; aa25 to aa32 of SEQ ID NO:11; aa26 to aa33 of SEQ ID NO:11; aa27 to aa34 of SEQ ID NO:11; aa28 to aa35 of SEQ ID NO:11; aa29 to aa36 of SEQ ID NO:11; aa30 to aa37 of SEQ ID NO:11; aa31 to aa38 of SEQ ID NO:11; aa32 to aa39 of SEQ ID NO:11; aa33 to aa40 of SEQ ID NO:11; aa34 to aa41 of SEQ ID NO:11; aa35 to aa42 of SEQ ID NO:11; aa36 to aa43 of SEQ ID NO:11; aa37 to aa44 of SEQ ID NO:11; aa38 to aa45 of SEQ ID NO:11; aa39 to aa46 of SEQ ID NO:11; aa40 to aa47 of SEQ ID NO:11; aa41 to aa48 of SEQ ID NO:11; aa42 to aa49 of SEQ ID NO:11; aa43 to aa50 of SEQ ID NO:11; aa44 to aa51 of SEQ ID NO:11; aa45 to aa52 of SEQ ID NO:11; aa46 to aa53 of SEQ ID NO:11; aa47 to aa54 of SEQ ID NO:11; aa48 to aa55 of SEQ ID NO:11; aa49 to aa56 of SEQ ID NO:11; aa50 to aa57 of SEQ ID NO:11; aa51 to aa58 of SEQ ID NO:11; aa52 to aa59 of SEQ ID NO:11; aa53 to aa60 of SEQ ID NO:11; aa54 to aa61 of SEQ ID NO:11; aa55 to aa62 of SEQ ID NO:11; aa56 to aa63 of SEQ ID NO:11; aa57 to aa64 of SEQ ID NO:11; aa58 to aa65 of SEQ ID NO:11; aa59 to aa66 of SEQ ID NO:11; aa60 to aa67 of SEQ ID NO:11; aa61 to aa68 of SEQ ID NO:11; aa62 to aa69 of SEQ ID NO:11; aa63 to aa70 of SEQ ID NO:11; aa64 to aa71 of SEQ ID NO:11; aa65 to aa72 of SEQ ID NO:11; aa66 to aa73 of SEQ ID NO:11; aa67 to aa74 of SEQ ID NO:11; aa68 to aa75 of SEQ ID NO:11; aa69 to aa76 of SEQ ID NO:11; aa70 to aa77 of SEQ ID NO:11; aa71 to aa78 of SEQ ID NO:11; aa72 to aa79 of SEQ ID NO:11; aa73 to aa80 of SEQ ID NO:11; aa74 to aa81 of SEQ ID NO:11; aa75 to aa82 of SEQ ID NO:11; aa76 to aa83 of SEQ ID NO:11; aa77 to aa84 of SEQ ID NO:11; aa78 to aa84 of SEQ ID NO:11; aa79 to aa86 of SEQ ID NO:11; aa80 to aa87 of SEQ ID NO:11; aa81 to aa88 of SEQ ID NO:11; aa82 to aa89 of SEQ ID NO:11; aa83 to aa90 of SEQ ID NO:11; aa84 to aa91 of SEQ ID NO:11; aa85 to aa92 of SEQ ID NO:11; aa86 to aa93 of SEQ ID NO:11; aa87 to aa94 of SEQ ID NO:11; aa88 to aa95 of SEQ ID NO:11; aa89 to aa96 of SEQ ID NO:11; aa90 to aa97 of SEQ ID NO:11; aa91 to aa98 of SEQ ID NO:11; aa92 to aa99 of SEQ ID NO:11; aa93 to aa100 of SEQ ID NO:11; aa94 to aa101 of SEQ ID NO:11; aa95 to aa102 of SEQ ID NO:11; aa96 to aa103 of SEQ ID NO:11; aa97 to aa104 of SEQ ID NO:11; aa98 to aa105 of SEQ ID NO:11; aa99 to aa106 of SEQ ID NO:11; aa100 to aa107 of SEQ ID NO:11 aa101 to aa108 of SEQ ID NO:11 aa102 to aa109 of SEQ ID NO:11; aa103 to aa110 of SEQ ID NO:11; aa104 to aa111 of SEQ ID NO:11; aa105 to aa112 of SEQ ID NO:11; aa106 to aa113 of SEQ ID NO:11; aa107 to aa114 of SEQ ID NO:11; aa108 to aa115 of SEQ ID NO:11; aa109 to aa116 of SEQ ID NO:11; aa110 to aa117 of SEQ ID NO:11; aa111 to aa118 of SEQ ID NO:11; aa112 to aa119 of SEQ ID NO:11; aa113 to aa120 of SEQ ID NO:11; aa114 to aa121 of SEQ ID NO:11; aa115 to aa122 of SEQ ID NO:11; aa116 to aa123 of SEQ ID NO:11; aa117 to aa124 of SEQ ID NO:11; aa118 to aa125 of SEQ ID NO:11; aa119 to aa126 of SEQ ID NO:11; aa120 to aa127 of SEQ ID NO:11; aa121 to aa128 of SEQ ID NO:11; aa122 to aa129 of SEQ ID NO:11; aa123 to aa130 of SEQ ID NO:11; aa124 to aa131 of SEQ ID NO:11; aa125 to aa132 of SEQ ID NO:11; aa126 to aa133 of SEQ ID NO:11; aa127 to aa134 of SEQ ID NO:11; aa128 to aa135 of SEQ ID NO:11; aa129 to aa136 of SEQ ID NO:11; aa130 to aa137 of SEQ ID NO:11; aa131 to aa138 of SEQ ID NO:11; aa132 to aa139 of SEQ ID NO:11; aa133 to aa140 of SEQ ID NO:11; aa134 to aa141 of SEQ ID NO:11; aa135 to aa142 of SEQ ID NO:11; aa136 to aa143 of SEQ ID NO:11; aa137 to aa144 of SEQ ID NO:11; aa138 to aa145 of SEQ ID NO:11; aa139 to aa146 of SEQ ID NO:11; aa140 to aa147 of SEQ ID NO:11; aa141 to aa148 of SEQ ID NO:11; aa142 to aa149 of SEQ ID NO:11; aa143 to aa150 of SEQ ID NO:11; aa144 to aa151 of SEQ ID NO:11; aa145 to aa152 of SEQ ID NO:11; aa146 to aa153 of SEQ ID NO:11; aa147 to aa154 of SEQ ID NO:11; aa148 to aa155 of SEQ ID NO:11; aa149 to aa156 of SEQ ID NO:11; aa150 to aa157 of SEQ ID NO:11; aa151 to aa158 of SEQ ID NO:11; aa152 to aa159 of SEQ ID NO:11; aa153 to aa160 of SEQ ID NO:11; aa154 to aa161 of SEQ ID NO:11; aa155 to aa162 of SEQ ID NO:11; aa156 to aa163 of SEQ ID NO:11; aa157 to aa164 of SEQ ID NO:11; aa158 to aa165 of SEQ ID NO:11; aa159 to aa166 of SEQ ID NO:11; aa160 to aa167 of SEQ ID NO:11; aa161 to aa168 of SEQ ID NO:11; aa162 to aa169 of SEQ ID NO:11; aa163 to aa170 of SEQ ID NO:11; aa164 to aa171 of SEQ ID NO:11; aa165 to aa172 of SEQ ID NO:11; aa166 to aa173 of SEQ ID NO:11; aa167 to aa174 of SEQ ID NO:11; aa168 to aa175 of SEQ ID NO:11; aa169 to aa176 of SEQ ID NO:11; aa170 to aa177 of SEQ ID NO:11; aa171 to aa178 of SEQ ID NO:11; aa172 to aa179 of SEQ ID NO:11; aa173 to aa180 of SEQ ID NO:11; aa174 to aa181 of SEQ ID NO:11; aa175 to aa182 of SEQ ID NO:11; aa176 to aa183 of SEQ ID NO:11; aa177 to aa184 of SEQ ID NO:11; aa178 to aa185 of SEQ ID NO:11; aa179 to aa186 of SEQ ID NO:11; aa180 to aa187 of SEQ ID NO:11; aa181 to aa188 of SEQ ID NO:11; aa182 to aa189 of SEQ ID NO:11; aa183 to aa190 of SEQ ID NO:11; aa184 to aa191 of SEQ ID NO:11; aa185 to aa192 of SEQ ID NO:11; aa186 to aa193 of SEQ ID NO:11; aa187 to aa194 of SEQ ID NO:11; aa188 to aa195 of SEQ ID NO:11; aa189 to aa196 of SEQ ID NO:11; aa190 to aa197 of SEQ ID NO:11; aa191 to aa198 of SEQ ID NO:11; aa192 to aa199 of SEQ ID NO:11; aa193 to aa200 of SEQ ID NO:11; aa194 to aa201 of SEQ ID NO:11; aa195 to aa202 of SEQ ID NO:11; aa196 to aa203 of SEQ ID NO:11; aa197 to aa204 of SEQ ID NO:11; aa198 to aa205 of SEQ ID NO:11; aa199 to aa206 of SEQ ID NO:11; aa200 to aa207 of SEQ ID NO:11; aa201 to aa208 of SEQ ID NO:11; aa202 to aa209 of SEQ ID NO:11; aa203 to aa210 of SEQ ID NO:11; aa204 to aa211 of SEQ ID NO:11; aa205 to aa212 of SEQ ID NO:11; aa206 to aa213 of SEQ ID NO:11; aa207 to aa214 of SEQ ID NO:11; aa208 to aa215 of SEQ ID NO:11; aa209 to aa216 of SEQ ID NO:11; aa210 to aa217 of SEQ ID NO:11; aa211 to aa218 of SEQ ID NO:11; aa212 to aa219 of SEQ ID NO:11; aa213 to aa220 of SEQ ID NO:11; aa214 to aa221 of SEQ ID NO:11; aa215 to aa222 of SEQ ID NO:11; aa216 to aa223 of SEQ ID NO:11; aa217 to aa224 of SEQ ID NO:11; aa218 to aa225 of SEQ ID NO:11; aa219 to aa226 of SEQ ID NO:11; aa220 to aa227 of SEQ ID NO:11; aa221 to aa228 of SEQ ID NO:11; aa222 to aa229 of SEQ ID NO:11; aa223 to aa230 of SEQ ID NO:11; aa224 to aa231 of SEQ ID NO:11; aa225 to aa232 of SEQ ID NO:11; aa226 to aa233 of SEQ ID NO:11; aa227 to aa234 of SEQ ID NO:11; aa228 to aa235 of SEQ ID NO:11; aa229 to aa236 of SEQ ID NO:11; aa230 to aa237 of SEQ ID NO:11; aa231 to aa238 of SEQ ID NO:11; aa232 to aa239 of SEQ ID NO:11; aa233 to aa240 of SEQ ID NO:11; aa234 to aa241 of SEQ ID NO:11; aa235 to aa242 of SEQ ID NO:11; aa236 to aa243 of SEQ ID NO:11; aa237 to aa244 of SEQ ID NO:11; aa238 to aa245 of SEQ ID NO:11; aa239 to aa246 of SEQ ID NO:11; aa240 to aa247 of SEQ ID NO:11; aa241 to aa248 of SEQ ID NO:11; aa242 to aa249 of SEQ ID NO:11; aa243 to aa250 of SEQ ID NO:11; aa244 to aa251 of SEQ ID NO:11; aa245 to aa252 of SEQ ID NO:11; aa246 to aa253 of SEQ ID NO:11; aa247 to aa254 of SEQ ID NO:11; aa248 to aa255 of SEQ ID NO:11; aa249 to aa256 of SEQ ID NO:11; aa250 to aa257 of SEQ ID NO:11; aa251 to aa258 of SEQ ID NO:11; aa252 to aa259 of SEQ ID NO:11; aa253 to aa260 of SEQ ID NO:11; aa254 to aa261 of SEQ ID NO:11; aa255 to aa262 of SEQ ID NO:11; aa256 to aa263 of SEQ ID NO:11; aa257 to aa264 of SEQ ID NO:11; aa258 to aa265 of SEQ ID NO:11; aa259 to aa266 of SEQ ID NO:11; aa260 to aa267 of SEQ ID NO:11; aa261 to aa268 of SEQ ID NO:11; aa262 to aa269 of SEQ ID NO:11; aa263 to aa270 of SEQ ID NO:11; aa264 to aa271 of SEQ ID NO:11; aa265 to aa272 of SEQ ID NO:11; aa266 to aa273 of SEQ ID NO:11; aa267 to aa274 of SEQ ID NO:11; aa268 to aa275 of SEQ ID NO:11; aa269 to aa276 of SEQ ID NO:11; aa270 to aa277 of SEQ ID NO:11; aa271 to aa278 of SEQ ID NO:11; aa272 to aa279 of SEQ ID NO:11; aa273 to aa280 of SEQ ID NO:11; aa274 to aa281 of SEQ ID NO:11; aa275 to aa282 of SEQ ID NO:11; aa276 to aa283 of SEQ ID NO:11; aa277 to aa284 of SEQ ID NO:11; aa278 to aa284 of SEQ ID NO:11; aa279 to aa286 of SEQ ID NO:11; aa280 to aa287 of SEQ ID NO:11; aa281 to aa288 of SEQ ID NO:11; aa282 to aa289 of SEQ ID NO:11; aa283 to aa290 of SEQ ID NO:11; aa284 to aa291 of SEQ ID NO:11; aa285 to aa292 of SEQ ID NO:11; aa286 to aa293 of SEQ ID NO:11; aa287 to aa294 of SEQ ID NO:11; aa288 to aa295 of SEQ ID NO:11; aa289 to aa296 of SEQ ID NO:11; aa290 to aa297 of SEQ ID NO:11; aa291 to aa298 of SEQ ID NO:11; aa292 to aa299 of SEQ ID NO:11; aa293 to aa300 of SEQ ID NO:11; aa294 to aa301 of SEQ ID NO:11; aa295 to aa302 of SEQ ID NO:11; aa296 to aa303 of SEQ ID NO:11; aa297 to aa304 of SEQ ID NO:11; aa298 to aa305 of SEQ ID NO:11; aa299 to aa306 of SEQ ID NO:11; aa300 to aa307 of SEQ ID NO:11 aa301 to aa308 of SEQ ID NO:11 aa302 to aa309 of SEQ ID NO:11; aa303 to aa310 of SEQ ID NO:11; aa304 to aa311 of SEQ ID NO:11; aa305 to aa312 of SEQ ID NO:11; aa306 to aa313 of SEQ ID NO:11; aa307 to aa314 of SEQ ID NO:11; aa308 to aa315 of SEQ ID NO:11; aa309 to aa316 of SEQ ID NO:11; aa310 to aa317 of SEQ ID NO:11; aa311 to aa318 of SEQ ID NO:11; aa312 to aa319 of SEQ ID NO:11; aa313 to aa320 of SEQ ID NO:11; aa314 to aa321 of SEQ ID NO:11; aa315 to aa322 of SEQ ID NO:11; aa316 to aa323 of SEQ ID NO:11; aa317 to aa324 of SEQ ID NO:11; aa318 to aa325 of SEQ ID NO:11; aa319 to aa326 of SEQ ID NO:11; aa320 to aa327 of SEQ ID NO:11; aa321 to aa328 of SEQ ID NO:11; aa322 to aa329 of SEQ ID NO:11; aa323 to aa330 of SEQ ID NO:11; aa324 to aa331 of SEQ ID NO:11; aa325 to aa332 of SEQ ID NO:11; aa326 to aa333 of SEQ ID NO:11; aa327 to aa334 of SEQ ID NO:11; aa328 to aa335 of SEQ ID NO:11; aa329 to aa336 of SEQ ID NO:11; aa330 to aa337 of SEQ ID NO:11; aa331 to aa338 of SEQ ID NO:11; aa332 to aa339 of SEQ ID NO:11; aa333 to aa340 of SEQ ID NO:11; aa334 to aa341 of SEQ ID NO:11; aa335 to aa342 of SEQ ID NO:11; aa336 to aa343 of SEQ ID NO:11; aa337 to aa344 of SEQ ID NO:11; aa338 to aa345 of SEQ ID NO:11; aa339 to aa346 of SEQ ID NO:11; aa340 to aa347 of SEQ ID NO:11; aa341 to aa348 of SEQ ID NO:11; aa342 to aa349 of SEQ ID NO:11; aa343 to aa350 of SEQ ID NO:11; aa344 to aa351 of SEQ ID NO:11; aa345 to aa352 of SEQ ID NO:11; aa346 to aa353 of SEQ ID NO:11; aa347 to aa354 of SEQ ID NO:11; aa348 to aa355 of SEQ ID NO:11; aa349 to aa356 of SEQ ID NO:11; aa350 to aa357 of SEQ ID NO:11; aa351 to aa358 of SEQ ID NO:11; aa352 to aa359 of SEQ ID NO:11; aa353 to aa360 of SEQ ID NO:11; aa354 to aa361 of SEQ ID NO:11; aa355 to aa362 of SEQ ID NO:11; aa356 to aa363 of SEQ ID NO:11; aa357 to aa364 of SEQ ID NO:11; aa358 to aa365 of SEQ ID NO:11; aa359 to aa366 of SEQ ID NO:11; aa360 to aa367 of SEQ ID NO:11; aa361 to aa368 of SEQ ID NO:11; aa362 to aa369 of SEQ ID NO:11; aa363 to aa370 of SEQ ID NO:11; aa364 to aa371 of SEQ ID NO:11; aa365 to aa372 of SEQ ID NO:11; aa366 to aa373 of SEQ ID NO:11; aa367 to aa374 of SEQ ID NO:11; aa368 to aa375 of SEQ ID NO:11; aa369 to aa376 of SEQ ID NO:11; aa370 to aa377 of SEQ ID NO:11; aa371 to aa378 of SEQ ID NO:11; aa372 to aa379 of SEQ ID NO:11; and aa373 to aa380 of SEQ ID NO:11. The coding sequence of fragments can be easily constructed by cleaving the unwanted nucleotides from the mutant or native human HR polypeptide coding sequences.

Fusions are fragment, mutant, or native human HR polypeptides with additional amino acids at either or both of the termini. The additional amino acid sequence is not necessarily homologous to sequence found in native hypothalmic receptor polypeptides. The additional amino acid residues can facilitate expression, detection, or activity of the polypeptide, for example. The additional amino acid sequence can also be used as linker to construct multimers of human HR polypeptides. The transmembrane domains or receptor loops from other seven transmembrane receptors can be fused with human HR polypeptides. All fusion polypeptides exhibit the desired immunological, ligand binding, or signal transduction properties.

At the minimum, an expression cassette will contain a promoter which is operable in the host cell and is operably linked to a human HR polypeptide coding sequence. Expression cassettes may also include signal sequences, terminators, selectable markers, origins of replication, and sequences homologous to host cell sequences. These additional elements are optional but can be included to optimize expression.

A promoter is a DNA sequence upstream or 5′ to the hypothalmic receptor polypeptide coding sequence to be expressed. The promoter will initiate and regulate expression of the coding sequence in the desired host cell. To initiate expression, promoter sequences bind RNA polymerase and initiate the downstream (3′) transcription of a coding sequence (e.g. structural gene) into mRNA. A promoter may also have DNA sequences that regulate the rate of expression by enhancing or specifically inducing or repressing transcription. These sequences can overlap the sequences that initiate expression. Most host cell systems include regulatory sequences within the promoter sequences. For example, when a repressor protein binds to the lac operon, an E. coli regulatory promoter sequence, transcription of the downstream gene is inhibited. Another example is the yeast alcohol dehydrogenase promoter, which has an upstream activator sequence (UAS) that modulates expression in the absence of a readily available source of glucose. Additionally, some viral enhancers not only amplify but also regulate expression in mammalian cells. These enhancers can be incorporated into mammalian promoter sequences, and the promoter will become active only in the presence of an inducer, such as a hormone or enzyme substrate (Sassone-Corsi and Borelli (1986) Trends Genet. 2:215; Maniatis et al. (1987) Science 236:1237).

Functional non-natural promoters may also be used, for example, synthetic promoters based on a consensus sequence of different promoters. Also, effective promoters can contain a regulatory region linked with a heterologous expression initiation region. Examples of hybrid promoters are the E. coli lac operator linked to the E. coli tac transcription activation region; the yeast alcohol dehydrogenase (ADH) regulatory sequence linked to the yeast glyceraldehyde-3-phosphate-dehydrogenase (GAPDH) transcription activation region (U.S. Pat. Nos. 4,876,197 and 4,880,734, incorporated herein by reference); and the cytomegalovirus (CMV) enhancer linked to the SV40 (simian virus) promoter.

A human HR polypeptide. coding sequence may also be linked in reading frame to a signal sequence. The signal sequence fragment typically encodes a peptide comprised of hydrophobic amino acids which directs the hypothalmic receptor polypeptide to the cell membrane. Preferably, there are processing sites encoded between the leader fragment and the gene or fragment thereof that can be cleaved either in vivo or in vitro. DNA encoding suitable signal sequences can be derived from genes for secreted endogenous host cell proteins, such as the yeast invertase gene (EP 12 873; JP 62,096,086), the A-factor gene (U.S. Pat. No. 4,588,684), interferon signal sequence (EP 60 057).

A preferred class of secretion leaders, for yeast expression, are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (typically about 25 to about 50 amino acid residues) (U.S. Pat. Nos. 4,546,083 and 4,870,008, incorporated herein by reference; EP 324 274). Additional leaders employing an alpha-factor leader fragment that provides for secretion include hybrid alpha-factor leaders made with a presequence of a first yeast signal sequence, but a pro-region from a second yeast alpha-factor. (See e.g., PCT WO 89/02463.). Mammalian secretion leaders can also be utilized, such tissue plasminogen activator.

Typically, terminators are regulatory sequences, such as polyadenylation and transcription termination sequences, located 3′ or downstream of the stop codon of the coding sequences. Usually, the terminator of native host cell proteins are operable when attached 3′ of the hypothalmic receptor polypeptide coding sequences. Examples are the Saccharomyces cerevisiae alpha-factor terminator and the baculovirus terminator. Further, viral terminators are also operable in certain host cells; for instance, the SV40 terminator is functional in CHO cells.

For convenience, selectable markers, an origin of replication, and homologous host cells sequences may optionally be included in an expression vector. A selectable marker can be used to screen for host cells that potentially contain the expression vector. Such markers may render the host cell immune to drugs such as ampicillin, chloramphenicol, erythromycin, neomycin, and tetracycline. Also, markers may be biosynthetic genes, such as those in the histidine, tryptophan, and leucine pathways. Thus, when leucine is absent from the media, for example, only the cells with a biosynthetic gene in the leucine pathway will survive.

An origin of replication may be needed for the expression vector to replicate in the host cell. Certain origins of replication enable an expression vector to be reproduced at a high copy number in the presence of the appropriate proteins within the cell. Examples of origins are the 2μ and autonomously replicating sequences, which are effective in yeast; and the viral T-antigen, effective in COS-7 cells.

Expression vectors may be integrated into the host cell genome or remain autonomous within the cell. Polynucleotide sequences homologous to sequences within the host cell genome may be needed to integrate the expression cassette. The homologous sequences do not always need to be linked to the expression vector to be effective. For example, expression vectors can integrate into the CHO genome via an unattached dihydrofolate reductase gene. In yeast, it is more advantageous if the homologous sequences flank the expression cassette. Particularly useful homologous yeast genome sequences are those disclosed in PCT WO90/01800, and the HIS4 gene sequences, described in Genbank, accession no. J01331.

The choice of promoter, terminator, and other optional elements of an expression vector will also depend on the host cell chosen. The invention is not dependent on the host cell selected. Convenience and the level of protein expression will dictate the optimal host cell. A variety of hosts for expression are known in the art and available from the American Type Culture Collection (ATCC). Bacterial hosts suitable for expressing an hypothalmic receptor polypeptide include, without limitation: Campylobacter, Bacillus, Escherichia, Lactobacillus, Pseudomonas, Staphylococcus, and Streptococcus. Yeast hosts from the following genera may be utilized: Candida, Hansenula, Kluyveromyces, Pichia, Saccharomyces, Schizosaccharomyces, and Yarrowia. Immortalized mammalian host cells include but are not limited to CHO cells, HeLa cells, baby hamster kidney (BHK) cells, monkey kidney cells (COS), human hepatocellular carcinoma cells (e.g., Hep G2), and other cell lines. A number of insect cell hosts are also available for expression of heterologous proteins: Aedes aegypti, Bombyx mori, Drosophila melanogaster, and Spodoptera frugiperda (PCT WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Transformation

After vector construction, the desired hypothalmic receptor polypeptide expression vector is inserted into the host cell. Many transformation techniques exist for inserting expression vectors into bacterial, yeast, insect, and mammalian cells. The transformation procedure to introduce the expression vector depends upon the host to be transformed.

Methods of introducing exogenous DNA into bacterial hosts are well-known in the art, and typically protocol includes either treating the bacteria with CaCl₂ or other agents, such as divalent cations and DMSO. DNA can also be introduced into bacterial cells by electroporation or viral infection. Transformation procedures usually vary with the bacterial species to be transformed. See e.g., (Masson et al. (1989) FEMS Microbiol. Lett. 60:273; Palva et al. (1982) Proc. Natl. Acad. Sci. USA 79:5582; EP Publ. Nos. 036 259 and 063 953; PCT WO 84/04541, Bacillus), (Miller et al. (1988) Proc. Natl. Acad. Sci. 85:856; Wang et al. (1990) J. Bacteriol. 172:949, Campylobacter), (Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids in Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia), (Chassy et al. (1987) FEMS Microbiol. Lett. 44:173.Lactobacillus); (Fiedler et al. (1988) Anal. Biochem 170:38, Pseudomonas); (Augustin et al. (1990) FEMS Microbiol. Lett. 66:203, Staphylococcus), (Barany et al. (1980) J. Bacteriol. 144:698; Harlander (1987) “Transformation of Streptococcus lactis by electroporation,” in Streptococcal Genetics (ed. J. Ferretti and R. Curtiss III); Perry et al. (1981) Infec. Immun. 32:1295; Powell et al. (1988) Appl. Environ. Microbiol. 54:655; Somkuti et al. (1987) Proc. 4th Evr. Cong. Biotechnology 1:412, Streptococcus).

Transformation methods for yeast hosts are well-known in the art, and typically include either the transformation of spheroplasts or of intact yeast cells treated with alkali cations. Electroporation is another means for transforming yeast hosts. See for example, Methods in Enzymology, Volume 194, 1991, “Guide to Yeast Genetics and Molecular Biology.” Transformation procedures usually vary with the yeast species to be transformed. See e.g., (Kurtz et al. (1986) Mol. Cell. Biol. 6:142; Kunze et al. (1985) J. Basic Microbiol. 25:141; Candida); (Gleeson et al. (1986) J. Gen. Microbiol. 132:3459; Roggenkamp et al. (1986) Mol. Gen. Genet. 202:302; Hansenula); (Das et al (1984) J. Bacteriol. 158:1165; De Louvencourt et al. (1983) J. Bacteriol. 154:1165; Van den Berg et al. (1990) Bio/Technology 8:135; Kluyveromyces); (Cregg et al. (1985) Mol. Cell. Biol. 5:3376; Kunze et al. (1985) J. Basic Microbiol. 25:141; U.S. Pat. Nos. 4,837,148 and 4,929,555; Pichia); (Hinnen et al. (1978) Proc. Natl. Acad. Sci. USA 75;1929; Ito et al. (1983) J. Bacteriol. 153:163 Saccharomyces); (Beach and Nurse (1981) Nature 300:706; Schizosaccharomyces); (Davidow et al. (1985) Curr. Genet. 10:39; Gaillardin et al. (1985) Curr. Genet. 10:49; Yarrowia).

Methods for introducing heterologous polynucleotides into mammalian cells are known in the art and include viral infection, dextran-mediated transfection, calcium phosphate precipitation, polybrene mediated transfection, protoplast fusion, electroporation, encapsulation of the polynucleotide(s) in liposomes, and direct microinjection of the DNA into nuclei.

The method for construction of an expression vector for transformation of insect cells for expression of recombinant herein is slightly different than that generally applicable to the construction of a bacterial expression vector, a yeast expression vector, or a mammalian expression vector. In an embodiment of the present invention, a baculovirus vector is constructed in accordance with techniques that are known in the art, for example, as described in Kitts et al., BioTechniques 14: 810-817 (1993), Smith et al., Mol. Cell. Biol. 3: 2156 (1983), and Luckow and Summer, Virol. 17: 31 (1989). In one embodiment of the present invention, a baculovirus expression vector is constructed substantially in accordance to Summers and Smith, Texas Agricultural Experiment Station Bulletin No. 1555 (1987). Moreover, materials and methods for baculovirus/insect cell expression systems are commercially available in kit form, for example, the MaxBac® kit from Invitrogen (San Diego, Calif.).

Also, methods for introducing heterologous DNA into an insect host cell are known in the art. For example, an insect cell can be infected with a virus containing an hypothalmic receptor polypeptide coding sequence. When the virus is replicating in the infected cell, the hypothalmic receptor polypeptide will be expressed if operably linked to a suitable promoter. A variety of suitable insect cells and viruses are known and include following without limitation.

Insect cells from any order of the Class Insecta can be grown in the media of this invention. The orders Diptera and Lepidoptera are preferred. Example of insect species are listed in Weiss et al., “Cell Culture Methods for Large-Scale Propagation of Baculoviruses,” in Granados et al. (eds.), The Biology of Baculoviruses: Vol. II Practical Application for Insect Control, pp. 63-87 at p. 64 (1987). Insect cell lines derived from the following insects are exemplary: Carpocapsa pomeonella (preferably, cell line CP-128); Trichoplusia ni (preferably, cell line TN-368); Autograph californica; Spodoptera frugiperda (preferably, cell line Sf9); Lymantria dispar, Mamestra brassicae; Aedes albopictus; Orgyia pseudotsugata; Neodiprio sertifer; Aedes aegypti; Antheraea eucalypti; Gnorimoschema operceullela; Galleria mellonella; Spodoptera littolaris; Blatella germanic; Drosophila melanogaster, Heliothis zea, Spodoptera exigua; Rachiplusia ou; Plodia interpunctella; Amsaeta moorei; Agrotis c-nigrum; Adoxophyes orana; Agrotis segetum; Bombyx mori; Hyponomeuta malinellu; Colias eurytheme; Anticarsia germmetalia; Apanteles melanoscelu; Arctia caja; and Porthetria dispar. Preferred insect cell lines are from Spodoptera frugiperda, and especially preferred is cell line Sf9. The Sf9 cell line used in the examples herein was obtained from Max D. Summers (Texas A & M University, College Station, Tex., 77843, U.S.A.) Other S. frugiperda cell lines, such as IPL-Sf-21AE III, are described in Vaughn et al., In Vitro 13: 213-217 (1977).

The insect cell lines of this invention are suitable for the reproduction of numerous insect-pathogenic viruses such as parvoviruses, pox viruses, baculoviruses and rhabdcoviruses, of which nucleopolyhedrosis viruses (NPV) and granulosis viruses (GV) from the group of baculoviruses are preferred. Further preferred are NPV viruses such as those from Autographa spp., Spodoptera spp., Trichoplusia spp., Rachiplusia spp., Gallerai spp., and Lymantria spp. More prefferred are baculovirus strain Autographa californica NPV (AcNPV), Rachiplusia ou NPV, Galleria mellonella NPV, and any plaque purified strains of AcNPV, such as E2, R9, S1, M3, characterized and described by Smith et al., J Virol 30: 828-838 (1979); Smith et al., J Virol 33: 311-319 (1980); and Smith et al., Virol 89: 517-527 (1978).

Typically, insect cells Spodoptera frugiperda type 9 (SF9) are infected with baculovirus strain Autographa californica NPV (AcNPV) containing an hypothalmic receptor polypeptide coding sequence. Such a baculovirus is produced by homologous recombination between a transfer vector containing the coding sequence and baculovirus sequences and a genomic baculovirus DNA Preferably, the genomic baculovirus DNA is linearized and contains a disfunctional essential gene. The transfer vector, preferably, contains the nucleotide sequences needed to restore the disfunctional gene and a baculovirus polyhedrin promoter and terminator operably linked to the hypothalmic receptor polypeptide coding sequence. (See Kitts et al., BioTechniques 14(5): 810-817 (1993).

The transfer vector and linearized baculovirus genome are transfected into SF9 insect cells, and the resulting viruses probably containing the desired coding sequence. Without a functional essential gene the baculovirus genome cannot produce a viable virus. Thus, the viable viruses from the transfection most likely contain the hypothalmic receptor polypeptide coding sequence and the needed essential gene sequences from the transfer vector. Further, lack of occlusion bodies in the infected cells are another verification that the hypothalmic receptor polypeptide coding sequence was incorporated into the baculovirus genome.

The essential gene and the polyhedrin gene flank each other in the baculovirus genome. The coding sequence in the transfer vector is flanked at its 5′ with the essential gene sequences and the polyhedrin promoter and at its 3′ with the polyhedrin terminator. Thus, when the desired recombination event occurs the hypothalmic receptor polypeptide coding sequence displaces the baculovirus polyhedrin gene. Such baculoviruses without a polyhedrin gene will not produce occlusion bodies in the infected cells. Of course, another means for determining if coding sequence was incorporated into the baculovirus genome is to sequence the recombinant baculovirus genomic DNA. Alternatively, expression of the desired hypothalmic receptor polypeptide by cells infected with the recombinant baculovirus is another verification means.

Monitoring Human Hypothalmic Receptor Polypeptide Expression Levels

Immunoassays and ligand binding assays can be utilized to determine if the transformed host cell is expressing the desired hypothalmic receptor polypeptide.

For example, an immunofluorescence assay can be easily performed on transformed host cells without separating the human HR polypeptides from the cell membrane. The host cells are first fixed onto a solid support, such as a microscope slide or microtiter well. This fixing step permeabilizes the cell membrane. Next, the fixed host cells are exposed to an anti-human HR polypeptide antibody. Preferably, to increase the sensitivity of the assay, the fixed cells are exposed to a second antibody, which is labelled and binds to the anti-hypothalmic receptor polypeptide antibody. Typically, the secondary antibody is labelled with an fluorescent marker. The host cells which express the human HR polypeptides will be fluorescently labelled and easily visualized under the microscope. See, for example, Hashido et al., Biochem & Biophys Res Comm 187(3): 1241-1248 (1992).

Also, the human HR polypeptides do not need to be separated from the cell membrane for ligand binding assay. The host cells may be fixed to a solid support, such as a microtiter plate. Alternatively, a crude membrane fraction can be separated from lysed host cells by centrifugation (See Adachi et al., FEBS Lett 311 (2): 179-183 (1992)). The fixed host cells or the crude membrane fraction is exposed to labelled, or other suitable ligand such as an agonist or antagonist. Typically, the ligand is labelled with radioactive atoms. The host cells which express the desired human HR polypeptide will bind with the labelled ligand which can be easily detected.

Purification

The purified hypothalmic receptor polypeptides are useful for signal transduction assays, ligand/receptor binding assays. The purified polypeptides can also be utilized to produce human HR polypeptide specific antibodies.

For ligand/receptor binding studies, the crude cell membrane fractions can be utilized. These membrane extracts can be isolated from cells which expressed hypothalmic receptor polypeptides by lysing the cells and separating the cell membrane fraction from the intracellular fractions by centrifugation. See Adachi et al., FEBS Lett 311(2): 179-183 (1992) for ligand binding assay procedure using cell membranes. Alternatively, whole cells, expressing hypothalmic receptor polypeptides, can be cultured in a microtiter plate, for example, and used for ligand binding assay.

Once the polypeptide has been dissociated from the cell membrane, the desired hypothalmic receptor polypeptide can also be affinity purified with specific hypothalmic receptor antibodies.

Antibodies

Antibodies against human HR polypeptides are useful for affinity chromatography, immunofluorescent assays, and distinguishing human from rat HR polypeptides

Such antibodies can be used to distinguish human from rat hypothalmic receptor polypeptides. These antibodies are useful in immunofluorescent assays when the cells are processed so that the membrane is made permeable. The permeablization of the cell membrane permits the antibodies to bind to cytoplasmic loops of the hypothalmic receptor polypeptides. Peptides containing the epitopes of interest can be easily synthesized using known automated synthesizer and gel purified for antibody production.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization.

Polyclonal antisera is obtained by bleeding the immunized animal into a glass or plastic container, incubating the blood at 25° C. for one hour, followed by incubating at 4° C. for 2-18 hours. The serum is recovered by centrifugation (e.g., 1,000×g for 10 minutes). About 20-50 ml per bleed may be obtained from rabbits.

Monoclonal antibodies are prepared using the method of Kohler and Milstein, Nature (1975) 256:495-96, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected MAb-secreting hybridomas are then cultured either in vitro (e.g. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Use in Biological Assays

Human HR polypeptides can also be used to screen peptide libraries to determine the amino acid sequence of agonist or antagonists.

A “library” of peptides may be synthesized following the methods disclosed in U.S. Pat. No. 5,010,175, and in PCT WO91/17823, both incorporated herein by reference in full. Briefly, one prepares a mixture of peptides, which is then screened to determine the peptides exhibiting the desired signal transduction and receptor binding activity. In the '175 method, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g., hexamers) is formed. Note that one need not include all amino acids in each step: one may include only one or two amino acids in some steps (e.g., where it is known that a particular amino acid is essential in a given position), thus reducing the complexity of the mixture. After the synthesis of the peptide library is completed, the mixture of peptides is screened for binding to the selected hypothalmic receptor polypeptide. The peptides are then tested for their ability to inhibit or enhance hypothalmic receptor signal transduction activity. Peptides exhibiting the desired activity are then isolated and sequenced.

The method described in '17823 is similar. However, instead of reacting the synthesis resin with a mixture of activated amino acids, the resin is divided into twenty equal portions (or into a number of portions corresponding to the number of different amino acids to be added in that step), and each amino acid is coupled individually to its portion of resin. The resin portions are then combined, mixed, and again divided into a number of equal portions for reaction with the second amino acid. In this manner, each reaction may be easily driven to completion. Additionally, one may maintain separate “subpools” by treating portions in parallel, rather than combining all resins at each step. This simplifies the process of determining which peptides are responsible for any observed receptor binding or signal transduction activity.

In such cases, the subpools containing, e.g., 1-2,000 candidates each are exposed to the desired hypothalmic receptor polypeptide. Each subpool that produces a positive result is then resynthesized as a group of smaller subpools (sub-subpools) containing, e.g., 20-100 candidates, and reassayed. Positive sub-subpools may be resynthesized as individual compounds, and assayed finally to determine the peptides, which exhibit a high binding constant. Then, these peptides can be tested for their ability to inhibit or enhance the HYPOTHALMIC signal transduction activity. The methods described in '17823 and U.S. Pat. No. 5,194,392 (herein incorporated by reference) enable the preparation of such pools and subpools by automated techniques in parallel, such that all synthesis and resynthesis may be performed in a matter of days.

Peptide agonists or antagonists are screened using any available method. The methods described herein are presently preferred. The assay conditions ideally should resemble the conditions under which the hypothalmic receptor signal transduction is exhibited in vivo, i.e., under physiologic pH, temperature, ionic strength, etc. Suitable agonists or antagonists will exhibit strong inhibition or enhancement of the hypothalmic signal transduction activity at concentrations which do not raise toxic side effects in the subject. Agonists or antagonists which compete for binding to the hypothalmic receptor ligand binding site may require concentrations equal to or greater than the hypothalmic receptor concentration, while inhibitors capable of binding irreversibly to the hypothalmic receptor may be added in concentrations on the order of the hypothalmic receptor concentration.

Signal Transduction Assays

Most cellular Ca²⁺ ions are sequestered in the mitochondria, endoplasmic reticulum, and other cytoplasmic vesicles, but receptor binding will trigger the increase of free Ca²⁺ ions in the cytoplasm With fluorescent dyes, such as fura-2, the concentration of free Ca²⁺ can be monitored. The ester of fura-2 is added to the media of the host cells expressing hypothalmic receptor polypeptides. The ester of fura-2 is lipophilic and diffuses across the membrane. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to the free Ca²⁺ ions, which are released after binding of a ligand to the hypothalmic receptor. The fluorescence can be measured without lysing the cells at an excitation spectrum of 340 nm or 380 nm and at fluorescence spectrum of 500 nm. See Sakurai et al., EP 480 381 and Adachi et al., FEBS Lett 311(2): 179-183 (1992) for examples of assays measuring free intracellular Ca²⁺ concentrations.

The rise of free cytosolic Ca²⁺ concentrations is preceded by the hydrolysis of phosphatidylinositol 4,5-bisphosphate. Hydrolysis of this phospholipid by the plasma-membrane enzyme phospholipase C yields 1,2-diacylglycerol (DAG), which remains in the membrane, and the water-soluble inosital 1,4,5-trisphosphate (IP₃). Binding of ligand or agonists will increase the concentration of DAG and IP₃. Thus, signal transduction activity can be measured by monitoring the concentration of these hydrolysis products.

To measure the IP₃ concentrations, radioactively labeled ³H-inositol is added to the media of host cells expressing hypothalmic receptor polypeptides. The ³H-inositol taken up by the cells and after stimulation of the cells with agonists,for example, the resulting inositol triphosphate is separated from the mono and di-phosphate forms and measured. See Sakurai et al., EP 480 381. Alternatively, Amersham provides an inosital 1,4,5-trisphosphate assay system. With this system Amersham provides tritylated inosital 1,4,5-trisphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc Natl Acad Sci 67: 305-312 (1970). In addition, a kit for assaying levels of cAMP is available from Diagnostic Products Corp. (Los Angeles, Calif., U.S.A.).

### C. EXAMPLES

The examples presented below are provided as a further guide to the practitioner of ordinary skill in the art, and are not to be construed as limiting the invention in any way.

### Example 1

Messenger RNA was isolated from human hypothalmic tissue using the mRNA isolation kit and instructions from Stratagene (La Jolla, Calif., U.S.A.). Random and oligo dt primed cDNA was made from RNA extracted from human hypothalamic tissue. The cDNA was made with the Superscript (trademark) pre amplification system kit by Gibco BRL according to the manufacturer's instructions (Gaithersburg, Md., U.S.A.).

Two degenerate oligonucleotide primers, named DO-42 and DO-43, were used in the PCR amplification. The sequence of DO-42 and DO-43 are as follows:

| | |
|---|---|
| DO-42: | ACAATATTAMAARIRIATGMGRAMLIGTIACSAAC |
| and | |
| DO-43: | ACAGGCCTflSAJRMAICMRTAIAWIATGGGRTFG. |

DO-42 contains a 5′ terminal SspI site and nucleotides encoding amino acids from the second transmembrane domain based on a consensus of seven transmembrane receptor sequences. DO-43 contains a 5′ terminal StuI site and the complement of nucleotides encoding amino acids from the seventh transmembrane domain based on a consensus of seven transmembrane receptor sequences.

PCR was performed using pooled CDNA (random and oligo dt primed) as template and DO-42 and DO-43 oligos as primers. Fifty picomoles of each oligo were used per reaction. Two 25 cycle rounds of PCR were performed using a melting temperature of 94 degree (c) for 30 seconds; 50 degree annealing temperature (40 degree second round) for 1 minute; and a 68 degree extension for 2 minutes. The resulting sample was run on a low melting point agarose gel. A region of the gel corresponding PCR products of size ranging from 600 to 900 nucleotides was cut from the gel.

One more round of PCR (25 cycles) was performed. The temperature cycle had a melting temperature of 94 degree for 30 seconds; 50 degree annealing for 1 minute; and a 68 degrees extension for 2 minutes on 5 microliters of melted gel. The resulting PCR products were subjected to gel electrophoresis. A band approximately 750 nucleotides is size was isolated and extracted from the gel. The purified DNA was then cloned and subjected to sequence analysis.

The nucleotide sequence of approximately 250 nucleotides is shown in SEQ ID NO:3 of the Sequence Listing. SEQ ID NO:1 is the putative amino acid sequence, which is encoded. The nucleotide sequence was included in a plasmid, DP254, deposited as a transformed E. coli strain with the ATCC.

The remaining nucleotide sequence encoding the complete native human HR can be isolated from cDNA or genomic libraries using the deposited plasmid as a probe. Various libraries are sold by Stratagene, for example. A human brain cDNA library is preferred. Such a library can be purchased from Stratagene (La Jolla, Calif., U.S.A.), catalog number 936213, as the Human Brain Library Lambda ZAP (registered) II Vector.

Radioactive probe can be constructed using the deposited plasmid, DP-254, and the rediprime kit by Amersham Life Science (Little Chalfont, Buckinghamshire, United Kingdom). The library can be probed and clones can be isolated according to the manufacturers' instructions included with the library, (Stratagene).

### Example 2

PCR was performed using as a template human genomic DNA from Promega, Madison Wis., U.S.A. The following primers were used in the reaction:

| | |
|---|---|
| DO-60: | CGGACTTTGATTACCTTTGAAC; |
| and | |
| DO-61: | TAAGTGGCATCAGATGACCAC. |

Fifty picomles of each primer was used and 324 nanograms of templates were used.

The reaction was first incubated at 94° C. for 2 minutes. Next, the reaction of 30 cycles were performed using (1) a melting temperature of 94° C. for 30 seconds, (2) an annealing temperature of 55° C. for 1 minute; and an extension temperature of 68° C. for 2 minutes. The reaction was terminated with a last incubation at 68° C. for 10 minutes.

From this reaction a band of ˜kb was isolated and inserted into a cloning vector. Four clones were isolated and the inserts were sequenced. The vectors were named HHR#13, HHR#14, HHR#1716, and HHR#18.

The sequence is shown as SEQ ID 6, 7,8, and 9. The consensus sequence is SEQ ID NO:10, the amino acid sequence of the consensus if SEQ ID NO:11. The clones were deposited with the ATCC as listed below.

Deposit Information:

The following materials were deposited with the American Type Culture Collection:

| | | Accession |
|---|---|---|
| Name | Deposit Date | No. |
| Escherichia coli DH5α, DP 254 | 29 Aug. 1995 | 69893 |
| Escherichia coli One Shot ™, HHR #13 | 23 Aug. 1996 | 98149 |
| Escherichia coli One Shot ™, HHR #14 | 23 Aug. 1996 | 98148 |
| Escherichia coli One Shot ™, HHR #1716 | 23 Aug. 1996 | 98151 |
| Escherichia coli One Shot ™, HHR #18 | 23 Aug. 1996 | 98150 |
| Phage Library 7.1 in M13LP67 | | 40828 |

The above materials have been deposited with the American Type Culture Collection, Rockville, Md., under the accession numbers indicated. This deposit will be maintained under the terms of the Budapest Treaty on the International Recognition of the Deposit of Microorganisms for purposes of Patent Procedure. The deposits will be maintained for a period of 30 years following issuance of this patent, or for the enforceable life of the paten, whichever is greater. Upon issuance of the patent, the deposits will be available to the public from the ATCC without restriction.

These deposits are provided merely as convenience to those of skill in the art, and are not an admission that a deposit is required under 35 U.S.C. §112. The sequence of the polynucleotides contained within the deposited materials, as well as the amino acid sequence of the polypeptides encoded thereby, are incorporated herein by reference and are controlling in the event of any conflict with the written description of sequences herein. A license may be required to make, use, or sell the deposited materials, and no such license is granted hereby.

What is claimed:

1. A polynucleotide comprising a human hypothalmic receptor (hHR) polypeptide coding sequence, wherein the receptor polypeptide comprises an amino acid sequence exhibiting more than 94% sequence identity to SEQ ID NO:11, and the polynucleotide is substantially free of other polynucleotides that do not encode hHR polypeptide.

2. The polynucleotide of claim 1, wherein the polynucleotide is a RNA or DNA molecule.

3. The polynucleotide of claim 2, wherein the polynucleotide is a cDNA molecule or genomic DNA.

4. The polynucleotide of claims 2, wherein the polynucleotide is obtainable as follows:
(a) isolating mRNA from human cells that contains hHR polypeptide;
(b) producing cDNA template therefrom;
(c) amplifying a portion of the the cDNA template using
a first polynucleotide primer, the sequence of the primers encodes at least three consecutive amino acids of SEQ ID NO:11 and using
a second polynucleotide primer, the reverse complement of the sequence of the second primer encodes at least three consecutive amino acids of SEQ ID NO:11,
wherein the first primer sequence is different from the second primer sequence; and
(d) obtaining the amplified polynucleotide fragment.

5. The polynucleotide of claim 2, wherein the polynucleotide is hybridizable under stringent condition to a sequence encoding a polypeptide comprising an amino acid sequence exhibiting more than 94% sequence identity to SEQ ID NO:1 or fragment thereof containing at least eight consecutive amino acids residues.

6. The polynucleotide of either claim 4 or 5, wherein the polynucleotide sequence is substantially the same as the sequence of claim 4 or 5, wherein the encoded amino acid sequence is unchanged.

7. The polynucleotide of claim 1, further comprising a promoter operably linked to said sequence encoding hHR polypeptide, wherein said promoter is either homologous or heterologous to the sequence.

8. The polynucleotide of claim 7, further comprising an origin of replication operably linked to said sequence encoding hHR polypeptide.

9. A human hypothalmic receptor polypeptide encoded by the polynucleotide of claim 1, wherein the polynucleotide is substantially free of intracellular proteins.

10. The polypeptide of claim 9, wherein said polypeptide exhibits an epitope not encoded by rat HR.

11. The polypeptide of claim 10, wherein said polypeptide is capable of modulating intracellular levels of inositol phosphate.

12. The polypeptide of claim 10, wherein said polypeptide is capable of modulating intracellular levels of Ca²⁺.

13. The polypeptide of claim 10, wherein said polypeptide is capable of modulating intracellular levels of diacylglycerol.

14. The polypeptide of claim 10, wherein said polypeptide is capable of modulating intracellular levels of cyclic AMP.

15. A polypeptide produced by the process comprising allowing the expression of the polypeptide in the cell having a polynucleotide of claim 1, and obtaining the expressed polypeptide therefrom.

16. The polypeptide of claim 15, wherein the cell is selected from the group consisting of a prokaryotic cell and an eukaryotic cell.

17. The polypeptide of claim 16, wherein the cell is selected from the group consisting of yeast, mammalian, inset, and avian.

18. A cell comprising a polynucleotide according to 1.

19. A method of producing human hypothalmic receptor polypeptide comprising:
(a) providing a cell having a polynucleotide of claim 1; and
(b) culturing said cell under conditions inducing production human hypothalmic receptor polypeptide.

20. A method of screening for a candidate capable of binding to a human hypothalmic receptor (HR) polypeptide comprising:
(a) providing a human BR polypeptide substantially free of other human intracellular components;
(b) exposing the human HR polypeptide to the candidate under conditions that permit the polypeptide and the candidate to bind and form a complex;
(c) measuring the amount of complex was formed.

21. A method A method of screening for a candidate triggering signal transduction activity of a human hypothalmic receptor (HR) polypeptide comprising:
(a) providing a human HR polypeptide substantially free of other human intracellular components;
(b) exposing the human HR polypeptide to the candidate under conditions that permit the polypeptide and the candidate to bind and form a complex;
(c) measuring the amount signal transduction activity.

22. A method of determining human hypothalmic receptor (HR) polypeptide signal transduction activity, wherein said method comprises
(a) providing a cell producing a human HR polypeptide;
(b) exposing said produced hHR polypeptide to a substrate;
(c) measuring hHR polypeptide signal transduction activity.

23. A method for detecting polynucleotides encoding an amino acid sequence exhibiting at least 80% sequence identity to SEQ ID NO:11, wherein said method comprises:
(a) providing a nucleic acid probe which hybridizes to SEQ ID NO:10;
(b) hybridizing a sample of polynucleotides to said probe to form a duplex; and
(c) detecting said duplexes.

24. An antibody capable of binding specifically to a human hypothalmic receptor polypeptide comprising an amino acid sequence exhibiting at least 80% sequence identity to SEQ ID NO:11 or fragment thereof.

25. The antibody of claim 18, wherein said antibody is capable of differentiating human hypothalmic receptor polypeptides from rat hypothalmic receptor.
